(19) United States
(12) Patent Application Publication
(10) Pub. No.: US 2017/0030907 A1
(43) Pub. Date: Feb. 2, 2017

(54) TLR-4 BASED ELECTROCHEMICAL BIOSENSOR

(71) Applicant: UTI Limited Partnership, Calgary (CA)

(72) Inventors: Robert Matthew Mayall, Calgary (CA); Margaret Mary Flora Renaud-Young, Calgary (CA); Viola Ingrid Birss, Calgary (CA)

(73) Assignee: UTI Limited Partnership, Calgary (CA)

(21) Appl. No.: 15/193,036

(22) Filed: Jun. 25, 2016

Related U.S. Application Data
(60) Provisional application No. 62/185,393, filed on Jun. 26, 2015.

(51) Int. Cl. G01N 33/569; G01N 27/02

(52) U.S. Cl. CPC G01N 33/56916; G01N 27/021; G01N 2333/70596; G01N 2333/255; G01N 2610/00

(57) ABSTRACT

The present invention relates to an apparatus comprising TLR-4 based electrochemical biosensor and a method for using the same to detect the presence of gram-negative bacteria or lipopolysaccharide in a sample. In one particular embodiment, the apparatus comprises an electric conducting solid substrate surface having a monolayer of a mixture of linkers each of which has a first functional group that is attached to the surface of said electric conducting solid substrate. The mixture of linkers comprises tethering-linkers and spacer-linkers such that the amount of spacer-linkers is equal to or greater than that of the tethering-linkers. The tethering linker also comprises a second functional group that is used to attach a toll-like receptor 4 (TLR-4).

### CROSS-REFERENCE TO RELATED APPLICATIONS

This application claims the priority benefit of U.S. Provisional Application No. 62/185,393, filed Jun. 26, 2015, which is incorporated herein by reference in its entirety.

### FIELD OF THE INVENTION

The present invention relates to an apparatus comprising TLR-4 based electrochemical biosensor and a method for using the same to detect the presence of gram-negative bacteria or lipopolysaccharide in a sample.

### BACKGROUND OF THE INVENTION

Infections affect millions of people each year, yet methods to ascertain their cause can take more than 24 hours to be effective. This delay between the presentation with symptoms and the ability to make an informed decision about treatment can have adverse consequences, including death in severe cases. Additionally, pathogen identification is a concern for public safety amid the growing threat of bioterrorism with the possibility of a terrorist organization releasing an infectious agent as an act of warfare. Developing a detection system based on the immune system offers the advantage of broad specificity, while still remaining pertinent to human health.

The current standard in the diagnosis of infections relies on slow, cell culture-based methodologies (Bloos, 2015). This relatively large amount of time taken to correctly identify a pathogenic agent is often detrimental (Bloos, 2015; Neu, 1992). Detection from airborne samples also suffers from these drawbacks, where the identification of bacteria, including Gram-negative bacteria, is slow.

Therefore, there is a need for a rapid and accurate method for identifying infectious agents, including from aerosol-based samples.

### SUMMARY OF THE INVENTION

Some aspects of the invention provide a method for using human Toll-Like Receptor-4 (TLR-4), a protein that can detect lipopolysaccharide (LPS), as a biosensor to detect the presence of a gram-negative bacteria or LPS in a sample. In some embodiments of the invention, TLR-4 is immobilized on a gold electrode via the tethering interaction of a modified Self-Assembled Monolayer (mSAM).

Still another aspect of the invention provides an electric conducting solid substrate surface having a monolayer of a mixture of linkers each of which has a first functional group that is attached to the surface of said electric conducting solid substrate, wherein said mixture of linkers comprises a tethering-linker and a spacer-linker in a ratio of x:1, wherein x is a number from 1 to 10, and wherein said tethering linker further comprises a second functional group; and a toll-like receptor 4 (TLR-4) that is attached to said tethering linker at said second functional group.

Another aspect of the invention provides a method for producing a solid substrate disclosed herein that comprises a surface bound TLR-4. In one particular embodiment of the invention, the method comprises:
(i) contacting an electric conducting solid substrate with a solution comprising a mixture of linkers each of which has a first functional group, said mixture of linkers comprising a tethering-linker and a spacer-linker in a ratio of x:y under conditions to form a monolayer of mixture of linkers wherein said first functional group is attached to the surface of said electric conducting solid substrate at a ratio of x:1 (where x is a number from 1 to 100; generally from 1 to 10, typically from 3 to 10 and often from 5 to 10) between said tethering-linker and said spacer-linker, and wherein said tethering-linker comprises a second functional group; and
(ii) attaching a toll-like receptor 4 (TLR-4) onto said second functional group of said tethering-linker.

One specific aspect of the invention provides a solid substrate comprising: an electric conducting solid substrate surface having a monolayer of a mixture of linkers each of which has a first functional group that is attached to the surface of said electric conducting solid substrate, wherein said mixture of linkers comprises a tethering-linker and a spacer-linker in a ratio of at least 1:1, and wherein said tethering linker comprises a second functional group; and a toll-like receptor 4 (TLR-4) that is attached to said tethering linker, wherein the chain length of said spacing-linker is smaller than the chain length of said tethering-linker.

In some embodiments, the ratio of said spacer-linker to said tethering-linker is at least 5:1. Yet in other embodiments, the chain length of said tethering-linker is at least 3 atoms longer than the chain length of said spacer-linker. Still in other embodiments, TLR-4 is attached to the tethering-linker through a metal cation that is coordinated to said second functional group. In one particular embodiment, the second function group of the tethering-linker comprises nitrilotriacetic acid (NTA), and the metal cation forms a metal-NTA coordinated complex, thereby allowing attachment of TLR-4 to the tethering-linker via a coordination of a polyhistidine group of the TLR-4 to the metal-NTA coordinated complex. In one particular instances, the metal comprises Ni⁺².

Yet in other embodiments, the TLR-4 further comprises lymphocyte antigen 96 (MD-2). In some instances, the TLR-4 is a recombinant human TLR-4/MD-2, that can optionally include a polyhistidine tag. A polyhistidine tag typically has at least 5, often at least 7, more often at least 10, and most often at least 15 consecutive histidine residues.

Another aspect of the invention provides a method for detecting the presence of a gram-negative bacteria in a sample. The method includes: (i) placing a sample in an apparatus comprising a solid substrate of the present invention under conditions sufficient to allow a gram-negative bacteria, if present in the sample, to attach to the solid substrate, in particular to the tethering-linker portion of the solid substrate; (ii) placing the resulting solid substrate in a redox solution; and (iii) measuring the impedance of the solid substrate to determine the presence of a gram-negative bacteria, where any significant change in impedance compared to a baseline impedance of the solid substrate is an indication that a gram-negative bacteria is present in the sample. The baseline impedance is typically impedance of the same or substantially similar solid substrate measured in the absence of any exposure to gram-negative bacteria or LPS.

Still another aspect of the invention provides a method for producing a solid substrate of the invention. The method includes contacting an electric conducting solid substrate with a solution comprising a mixture of linkers. Each linkers has a first functional group that is used to attach to the surface of a solid substrate. The mixture of linkers include a tethering-linker and a spacer-linker in a ratio of x:1. This mixture of linkers, typically in a solution, is contacted with the solid substrate under conditions sufficient to form a monolayer of mixture of linkers on the surface of the solid substrate. Once the linkers are attached to the surface of the solid substrate, a toll-like receptor 4 (TLR-4) is attached to a second functional group of the tethering-linker. In some instances, the second functional group of the tethering-linker is converted to a polyhistidine coordinating complex prior to attaching TLR-4. One specific example of the polyhistidine coordinating complex is a Ni-NTA complex which has been shown to coordinate with various polyhistidine molecules.

### BRIEF DESCRIPTION OF THE DRAWINGS

FIG. 1 shows a schematic illustration of one embodiment of the invention. In particular, panel (A) is a schematic of a modified SAM (mSAM) capable of immobilizing poly-histidine tagged proteins, such as TLR-4. Layer 1 is a mixed SAM of short and long alkanethiols attached to Au through a sulfur linkage, while Layer 2 consists of coordinated Ni2+ ions (Layer 3), which can then bind to a poly-histidine tag. Panel (B) is a schematic representation of the sensor bonded to TLR-4 (Layer 4), where the TLR-4 structure is adapted from the PDB file 3FXI. And Panel (C) is a schematic diagram showing how binding of LPS to TLR-4 causes a dimerization event that limits redox probe access to underlying Au surface.

FIG. 2 is a graph showing EIS response from SAM modified Au electrodes. Trace ‘a’ represents the response to an 11-mercaptoundecanoic acid SAM, while the inset shows the response to the (‘b’) 1-pentanethiol SAM and (‘c’) to a 1-propanethiol SAM. The equivalent circuit typically used for fitting the impedance data in this study to allow accurate determination of Rp is also shown. Rs represents the solution resistance, with Rp representing the resistance across the electrode interface and CPE representing a constant phase element.

FIG. 3 shows EIS response from a mSAM-based TLR-4 sensor composed of a 9:1 molar ratio of C3 spacer thiols:MUA. The impedance of the electrode increases as the concentration of LPS is increased from zero (TLR-4 baseline, trace A) and then 1 ng/mL LPS (trace B), to 10 μg/mL (trace F). All of the data were collected at the E0′ of the Fe2+/3+ couple in 0.2 M pH 7 PBS+5 mM Fe3+.

FIG. 4 is a graph showing percent increase in Rp of TLR-4 modified mSAM electrode to LPS vs. the baseline resistance of TLR-4 modified mSAMs with no LPS present. All mSAMs were constructed using a 9:1 molar ratio of C3 spacer:MUA. The resistance was calculated using the circuit-fitting program in the EC-Lab software, with the data fitted to the circuit shown in FIG. 2. Error bars represent the standard error of the mean with n=5.

FIG. 5 is a graph showing EIS response to gram-negative organisms of mSAM-based TLR-4 sensors composed of a 9:1 molar ratio of 1-propanethiol to MUA. The electrode responds reliably to increasing concentrations of S. typhimurium. Trace A represents the TLR-4 modified baseline, while trace B is for 100 cells/mL, increasing logarithmically to 10⁵ cells/mL in trace G. All data were collected at the E0′ of Fe2+/3+ in deaerated, non-stirred, 0.2 M pH 7 PBS+5 mM Fe3+.

FIG. 6A is a graph of representative EIS response from mSAM-based TLR-4 sensors composed of a 9:1 molar ratio of 1-propanethiol:11-mercaptoundecanoic acid to non-target organisms, namely heat killed S. aureau. All data were collected at the E0′ of Fe2+/3+ in deaerated, non-stirred, 0.2 M pH 7 PBS+5 mM Fe3+.

FIG. 6B is a graph of representative EIS response from mSAM-based TLR-4 sensors composed of a 9:1 molar ratio of 1-propanethiol:11-mercaptoundecanoic acid to non-target organisms, namely UV-inactivated viruses. All data were collected at the E0′ of Fe2+/3+ in deaerated, non-stirred, 0.2 M pH 7 PBS+5 mM Fe3+.

FIG. 7 is a graph of impedance response of a TLR-4 modified Au microelectrode (0.002 cm²) when exposed to various concentrations of heat-killed Salmonella cells (a gram-negative bacteria). Trace (a) represents the TLR-4 baseline before the addition any cells, (b) after addition of 10¹ cells/mL, (c) after addition of 10² cells/mL and (d) after addition of 10³ cells/mL.

FIG. 8 is a graph of EIS response of physisorbed TLR-4 on a clean (no SAM) Au substrate. The line b represents the EIS response of the electrode in phosphate buffer solution, while line a was obtained in a phosphate buffer solution that was spiked with 1 μg/mL LPS. All data were collected at the E0′ of Fe3+ in 0.2 M pH 7 PBS supplemented with 5 mM Fe3+.

FIG. 9 shows both random adsorption and orientated tethering of substrates as well as a graph of EIS data. In particular, FIG. 9 panel (A) is a schematic illustration of random adsorption of TLR-4 onto a SAM-modified electrode via amide bonds. Panel (B) is a schematic illustration showing the orientated tethering of TLR-4 onto a mSAM. And panel (C) shows a graph of EIS data obtained for a randomly oriented TLR-4 on a SAM modified electrode versus the TLR-4 baseline (trace a) when exposed to increasing concentrations of LPS from (b-f) 1, 10, 100, 1,000, and 10,000 ng/mL LPS. All data were collected at the E^{0′} of Fe^{2+/3+} in deaerated, unstirred, 0.2 M pH 7 PBS supplemented with 5 mM Fe³⁺.

FIG. 10 shows two exemplary methods for collecting air sample for bacteria detection using the sensor of the present invention.

### DETAILED DESCRIPTION OF THE INVENTION

Some aspects of the invention provide a sensor and a method that utilizes interaction between a complex of Toll-Like Receptor-4 (TLR-4) and lymphocyte antigen 96 (MD-2) with a gram-negative bacterial membrane component, lipopolysaccharide (LPS). TLR-4 and MD-2, hereafter referred simply as TLR-4, are part of the human innate immune system and are responsible for triggering the initial response when a gram-negative infection is identified or detected. See, for example, Beutler, 2000; Park et al., 2009. Some embodiments of the invention utilize electrochemistry to incorporate this biological interaction in a functional sensor as discussed herein. The apparatus of the invention can be used to inform the user of the presence and/or identity of gram-negative bacteria in a sample. In this manner, one can aid the decision of clinicians thereby potentially reducing the overuse of broad-spectrum antibiotics, which are not effective to gram-negative bacteria. In addition, apparatus of the invention can be used to detect presence of gram-negative bacteria in an air sample, for example, in environment, in hospitals, war fronts, etc.

One of the major challenges in electrochemical sensor development is achieving a reproducibly high concentration and sensitivity of ligand binding elements on the surface of the electrode. To overcome this shortcoming, the present inventors compared sensors produced from a range of modified Self Assembled Monolayers (mSAMs). SAMs are organic molecules, typically terminated with a sulfur moiety that form ordered structures on a gold substrate (Canaria et al., 2006; Gronbeck et al., 2000; Love et al., 2005). The synthesis of mSAMs can be achieved by the chemical modification of a SAM once it is bound to a substrate (Nicosia and Huskens, 2014). While some large and complex SAMs can interact directly with proteins, their synthesis and purification can be time consuming and costly (Han et al., 2006; Kroger et al., 1999).

The use of SAMs and mSAMs in biosensors is not new, with many previous sensing strategies already published (Amini et al., 2014; Besant et al., 2013; Das and Kelley, 2013; Ding et al., 2007; Han et al., 2006; Ivanov et al., 2013; Priano et al., 2007; Yeo et al., 2011). One differentiating factor in SAM-based sensors is the signal transduction method utilized. Common methods include voltammetry (Besant et al., 2013; Das and Kelley, 2013; Han et al., 2006; Ivanov et al., 2013), amperometry (Priano et al., 2007), and impedance spectroscopy (EIS) (Amini et al., 2014; Ding et al., 2007). The choice of technique is partially influenced by the magnitude of the resistance of the modified electrode, with low impedance sensors utilizing voltammetry and amperometry and high impedance devices focusing more on EIS.

Proteins have been immobilized on a Au microelectrode via a SAM. See, for example, Yeo et al., 2011. However, such a system lacked sufficient accuracy and/or sensitivity to be used as a biosensor.

The present inventors have discovered that some of the short comings of previous methods was at least in part due to the orientation of the protein. Accordingly, some aspects of the invention provide controlling the orientation of TLR-4 on the surface and the use of a mixture of SAMs of varying chain length. It should be noted other functional moieties have also been immobilized on electrode surfaces to detect LPS, most notably polymyxin B (Abdul Rahman et al., 2013; Ding et al., 2007; Iijima et al., 2011; Kato et al., 2007), an antibiotic shown to bind to LPS, and recently, DNA aptamers (Kim et al., 2012; Su et al., 2013, 2012), chosen for their LPS selectivity. While these sensors have achieved quite promising initial results, their response time and specificity, as well as the detection limits of bacterial cells, have been questionable to date.

By combining a naturally evolved sensing moiety, i.e., TLR-4, with the speed and portability of well-defined electrochemical techniques, the present invention provides a sensor capable of rapidly detecting LPS or bacteria in a fluid sample. Without begin bound by any theory, it is believed that the TLR-4 on the electrode surface of the present invention are oriented similarly to the conformation on the surface of a human cell. Such an orientation afforded mimicking the response and selectivity of the human immune system and detection of gram-negative bacteria over a biologically relevant range of concentrations. In particular, as discussed in more detail below, a proper orientation of TLR-4 in the sensors of invention is achieved by attaching the polyhistidine portion of TLR-4 to the SAM.

Another aspect of the invention provides a method for detecting the presence of a gram-negative bacteria (or LPS) in a sample. Such a method comprises:
(i) placing a sample, typically a fluid sample, and often a liquid sample, in an apparatus comprising:
   (a) an electric conducting solid substrate surface having a monolayer of a mixture of linkers each of which has a first functional group that is attached to the surface of said electric conducting solid substrate, wherein each of said mixture of linkers comprises a tethering linker interdispersed within spacing linkers, and wherein said tethering linker comprises a second functional group;
   (b) a toll-like receptor 4 (TLR-4) that is attached to said tethering linker; and
   (c) a solution of a redox active probe; and
(ii) measuring the electrical property within said apparatus to determine the presence of a gram-negative bacteria.

The terms “redox active probe” and “redox probe” are used interchangeably herein and refer to a probe that can be used to measure at least one electrochemical property of the substrate, e.g., such as reduction or oxidation potential, impedance, circular voltammetry, current, voltage, or any other electroproperties known to one skilled in the art.

Any type of electrical property that can be measured can be used to determine the presence of a gram-negative bacteria. Exemplary electrical properties that can be used include impedance, current (e.g., at a set voltage such as cyclic voltammetry), voltage (at a set current), or any other electrical properties that can be measured and known to one skilled in the art. In one particular embodiment, impedance is used to determine the presence of a gram-negative bacteria.

Typically, the electric conducting solid substrate comprises gold as the electric conducting substrate. Other suitable electric conducting solid substrates include, but are not limited to, platinum, silver, indium-tin oxide, silica, carbon, etc. It should be appreciated that the solid substrate can also include other materials such as a non-electric conducting substrates including, but not limited to, silicon wafer, glass, plastic, etc. The key element of the solid substrate is that the surface be covered with an electrically conductive substrate such as gold.

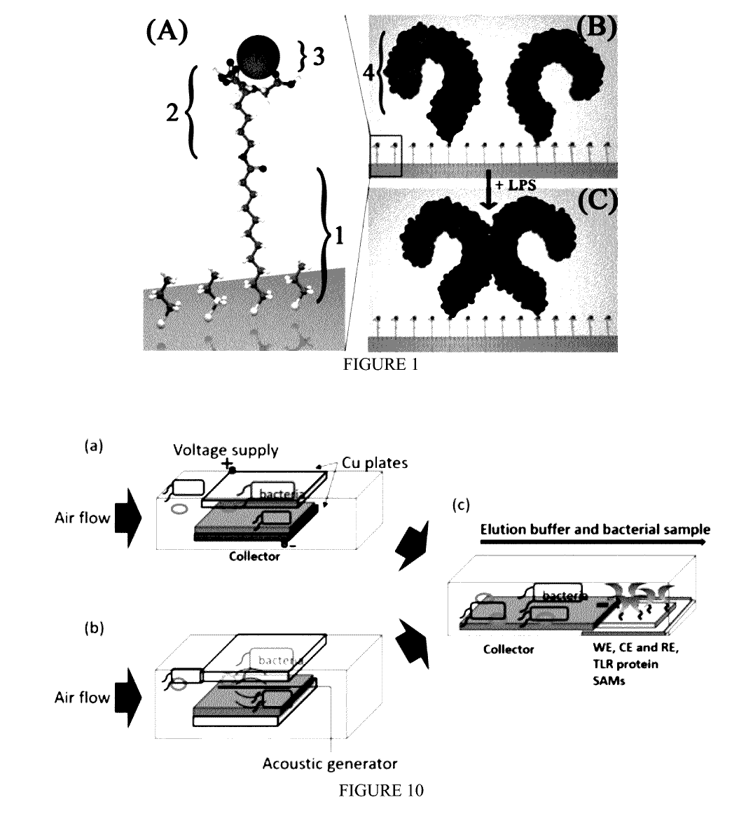

The mixture of linkers typically includes spacer-linkers and tethering-linkers. The spacer-linkers have shorter length compared to the tethering-linkers such that TLR-4's are bound to the tethering-linkers and is presented above the spacer-linkers. Generally, spacer-linkers comprise a chain of atoms of about 7 or less, typically 5 or less, and often 3 or less. As used herein, the term “chain of atoms” refers to number of non-hydrogen atoms (e.g., carbon, oxygen, nitrogen, etc.) and excludes the atom that attaches to the solid substrate surface. The number of atoms is counted from the atom that attaches to the solid substrate surface. Thus, for example, 1-propyl thiol is considered to have 3 chain of atoms when the sulfur is attached to a gold substrate, similarly 2-butyl thiol is also considered to have 3 chain of atoms since the thiol group that is attached to gold surface is located in the 2-position. The tethering-linker has longer chain of atoms than that of the spacer-linkers. Generally, the tethering-linker has at least 3, typically at least 5, and often at least 7 more chain of atoms compared to that of the spacer-linker. In this manner, the tethering-linker can be considered to allow presentation of TLR-4's “above” the surface of spacer-linkers as schematically illustrated in FIG. 1.

In some embodiments, the ratio of spacer-linkers to the tethering-linker is at least 1:1, typically at least 3:1, often at least 5:1, more often at least 7:1, and most often at least 9:1. In this manner, TLR-4's are spaced apart from one-another. It should be appreciated that the ratio simply refers to the ratio of the spacer-linkers and the tethering-linkers used to prepare the solid substrate. Without being bound by any theory, it is believed that use of such a ratio typically results in a statistical amount of separation between each tethering-linkers. However, it should be appreciated that it is most likely that some tethering-linkers will be spaced further apart and some tethering-linkers will be closer together. Thus, the ratio referred to herein merely refers to the ratio used to prepare such a substrate.

In contrast to conventional methods, in some embodiments, TLR-4 is attached to the tethering-linker in an orderly manner. The term “orderly manner” refers to having a particular portion (e.g., polyhistidine portion) of the TLR-4 being attached to the tethering-linker. Typically, at least 80%, often at least 85%, more often at least 90%, and most often at least 95% of TLR-4 is attached to the tethering-linker in a similar manner, e.g., polyhistidine portion of TLR-4 is attached to the tethering-linker. Alternatively, the term “orderly manner” refers to having a particular portion (e.g., polyhistidine or polycysteine portion) of the TLR-4 being attached to the tethering linker. It should also be appreciated that other “tags” can also be used to achieve this binding (e.g., streptavidin-biotin, FLAG tag, etc.). Without being bound by any theory, it is believed that this has the effect of orienting the TLR-4 to enhance the ability of the TLR-4 to interact with a particular ligand of the TLR-4. Again without being bound by any theory, it is believed that this typically involves orienting the ligand binding domain towards the solution and, most often, involves orienting the ligand binding domain towards the solution and the dimerization domains laterally across the electrode surface.

In some embodiments, the spacer-linker has only a first functional group that is used to attach to the solid substrate surface. While the spacer-linker can have a second functional group, it is believed that even if a second-functional group is present, due to the length of the tethering-linker being greater than the spacer-linker, majority (i.e., more than 50%, typically at least about 60%, often at least about 75%, more often at least about 80%, still more often at least 85%, and most often at least 90%), if not all, of TLR-4's will be bound to the tethering-linker. The term “about” when referring to a numeric value means±20%, typically ±10%, and often ±5% of the numeric value.

The tethering-linker includes a second functional group. Typically, the second functional group is present at the opposite end of the chain length from the first functional group. The first functional group is used to attach the tethering-linker to the solid substrate surface while the second functional group is used to attach TLR-4's.

Exemplary first functional groups include thiol, hydroxyl, amine, amide, carboxyl, etc. The linkers (spacer-linker and/or tethering-linker) can be bound to the solid substrate surface via an ion-bond, covalent-bond or simply metal-heteroatom (e.g., S, O or N) bond. Typically, for a gold substrate surface, thiol (e.g., S heteroatom) is used as the first functional group.

Exemplary second functional groups include nitrilotriacetic acid moiety, and ethylenediaminetetraacetic acid, ethylene glycol tetraacetic acid, and other polycarboxylic acid capable of chelating ions.

In some embodiments, a metal cation is attached to or forms a coordinating complex with the second functional group. Suitable metal cations include nickel, and copper, iron, calcium, cobalt, cadmium, mercury, silver, etc. In some instances, the metal cation is used to attach TLR-4 in a proper orientation. For example, nickel ion complex has been shown to attach or form a complex with the polyhistidine rich region of TLR-4. By attaching/binding or forming a complex with TLR-4 in the polyhistidine rich region, it is believed that the proper orientation of TLR-4 for presentation to bind to LPS or bacteria is achieved.

The sensors of the invention show response to varying concentrations of LPS and bacteria. In particular, the protein-electrode combination (i.e., TLR-4/LPS combination) showed a logarithmically proportional increased resistance to charge transfer due to the formation of TLR-4 protein dimers. It also demonstrated excellent sensitivity to trace levels of gram-negative bacteria, while remaining substantially completely insensitive (i.e., <5%, typically <1% and often <0.5% sensitivity) to both gram-positive and viral challenges. Further characterization of revealed that maintaining an orientation mimicking TLR-4's role in a cellular context resulted in the most responsive sensor.

The present invention will be further described with regard to the accompanying drawings which assist in illustrating various features of the invention. However, it should be appreciated that the scope of the invention is not limited to those described herein as one skilled in the art having read the present disclosure can readily modify the various elements of the invention.

As illustrated in FIG. 1, for the first layer of mSAM (FIG. 1A, single or mixed component SAM, referred to as Layer 1), a thiol-containing carboxylic acid, 11-mercaptoundecanoic acid, was utilized due to the Au-thiol interaction (Canaria et al., 2006; Gronbeck et al., 2000; Love et al., 2005). Other SAM designs involving short spacer thiols were also explored to reduce the overall impedance of the SAMs to levels tolerable by conventional electrochemical instruments. In one embodiment of mixed SAMs, the carboxylic acids were spaced by using a 9:1 molar ratio of the small spacer thiol, typically 1-propanethiol, to the 11-carbon carboxylate alkanethiol.

The carboxyl group on the outer surface of 11-carbon component of the SAM, exposed to solution, was then activated using previously published protocols to allow the formation of amide bonds (Bonroy et al., 2006; Witt and Klajn, 2004). This allowed tethering of the well-characterized nitrilotriacetic acid (NTA) group (FIG. 1A, Layer 2) to the modified electrode surface through an amide bond (Han et al., 2006; Kröger et al., 1999). Then by complexing Ni²⁺ to NTA (FIG. 1A, Layer 3), the Ni-NTA functionalized surface can effectively bind to proteins that have a poly-histidine tag (Han et al., 2006; Hochuli et al., 1987; Kroger et al., 1999). This allowed the attachment of any poly-histidine tagged protein (FIG. 1B, Layer 4), e.g., TLR-4, to the electrode in an ordered manner, and the subsequent examination of its properties electrochemically.

Without being bound by any theory, it is believed that the mode of action by which TLR-4 binds to LPS in a cellular context is through dimerization (Beutler, 2000; Park et al., 2009), where two TLR-4 molecules transiently bond together to trap two LPS molecules. It is believed that the method of invention also involves dimerization of proximal TLR-4s that results in modulation of the access of redox active ions to the underlying Au (i.e., gold) surface (FIG. 1C). As LPS or bacteria binds to the TLR-4 moieties on the electrode surface, access by probe redox-active ions in solution to the Au electrode is inhibited, as they are now only able to react at defects in the mSAM. See, for example, Campuzano et al., 2006; Diao et al., 2001; Han et al., 2006. Suitable redox probes (i.e., redox active probes) include, but are not limited to, (i) redox ions such as Fe³⁺, Fe²⁺, Ni²⁺, Ru²⁺, Ru³⁺, Cu²⁺, etc., (ii) organic redox probes such as hydroquinone, benzoquinone, para-aminophenol, para-iminoquinone, methane, CO₂, (iii) as well as other electrochemically active species including, but not limited to, oxygen, hydrogen, and other electrochemically active species known to one skilled in the art. It should be appreciated that redox ions are generally added as a salt. For example, exemplary Fe⁺² and Fe⁺³ redox ions include Fe(CN)₆⁴⁻, Fe(CN)₆³⁻, as well as other soluble ferrous and ferric salts. In one particular embodiment, a redox ion is used as a redox probe. In one specific instance, Fe⁺³ is used as a redox ion.

In one particular embodiment, the presence of the redox probe in solution, such as Fe³⁺, the impedance of solid substrate was monitored in a rapid and quantifiable manner. As more analyte (i.e., LPS or bacteria) was selectively bound to the TLR-4 groups on the electrode surface, the access to the underlying Au was further restricted, thus generating a gradually increasing impedance with increasing analyte concentration in the medium. It was shown that such a system (FIG. 1) leads to impedance changes that reflect the predicted TLR-4/LPS or TLR-4/gram-negative bacteria interactions, providing a rapid method of monitoring the presence of LPS, and gram-negative bacteria, in contaminated samples.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### Examples

Equipment & Electrodes: A standard three-electrode setup was used in conjunction with a Bio-Logic SP-300 potentiostat with an ultra-low current cable and impedance module for all electrochemical experiments, with the data being collected by the EC-Lab software (version 10.37). All experiments were performed inside a Faraday cage, with the exception of the Au surface cleaning cycles. All water used in this work was triply distilled prior to use and all experiments were performed at room temperature.

Au electrodes were purchased from Deposition Research Laboratories as sputtered glass slides with a 40 nm Ti adhesion layer and 100 nm of Au on top. The electrodes were rinsed in acetone, isopropanol, ethanol, and then water before being electrochemically cleaned in unstirred 0.5 M H₂SO₄ (EMD Millipore, ACS grade). For the electrochemical cleaning step, a three electrode setup was used with a platinum (Pt) mesh counter electrode (CE) and a reversible hydrogen reference electrode (RHE). The Au was electrically connected to a copper (Cu) clip that remained suspended above the solution. The potential of the Au-coated slides was scanned between 0.05 V and 1.7 V vs RHE at a sweep rate of between 100 mV/s and 500 mV/s until the CVs gave the characteristic response of Au. The H₂SO₄ was deaerated with nitrogen gas for 20 minutes prior to cleaning.

SAM Deposition & Testing: Self-assembled monolayers (SAMs) were formed on clean Au electrodes by submerging them in ethanolic solutions of 10 mM thiol for 24 hours. 11-mercaptoundecanoic acid (MUA, Sigma-Aldrich, 95% purity) was dissolved in ethanol to the desired concentration, while 1-propanethiol (Sigma-Aldrich, 99% purity) and 1-pentanethiol (Sigma-Aldrich, 99% purity) were diluted in ethanol. For mixed SAM construction, 1-propanethiol or 1-pentanethiol was diluted to 9 mM and combined with 1 mM MUA, achieving a total thiol concentration of 10 mM. After thiol attachment, the electrodes were rinsed sequentially with ethanol and then water before being evaluated electrochemically, using a Pt mesh counter electrode and an Ag/AgCl reference electrode.

A 5 mM solution of sodium salt of ferric ethylenediaminetetraacetic acid (Fe-EDTA, Sigma-Aldrich, BioReagent grade) was used as a redox probe and all EIS experiments were performed at the E^{0′} of the Fe(II/III)-EDTA couple. The E^{0′} was determined experimentally using a Pt microelectrode before each EIS experiment. The supporting electrolyte was 0.2 M pH 7 phosphate buffer solution that was vigorously bubbled with N₂ gas for 20 minutes to deaerate the cell solution prior to testing, after which N₂ was passed continuously over the solution surface.

Modified Self Assembled Monolayer (mSAM) Construction: The terminal COOH groups of 11-mercaptoundecanoic acid were activated by submerging the SAM-coated electrode in a solution of 2 mg 1-ethyl-3-(3-dimethylaminopropyl)carbodiimide (EDC, Sigma-Aldrich, 99% purity), 5.5 mg N-hydroxysuccinimide (NHS, Sigma-Aldrich, 98% purity), and 0.108 g 4-morphilineethanesulfonic acid (MES, Sigma-Aldrich, Biotechnology grade) in 5 mL of water for one hour under constant N₂ flow. Following the activation of the COOH groups, the electrode was submerged in 1 mM Nα,Nα-bis(carboxymethyl)-L-lysine (Sigma-Aldrich, 97% purity) at 4° C. for 24 hours. The electrode was then rinsed with water and soaked in 0.1 M Ni SO₄ for 15 minutes before being rinsed with water again. It was then submerged in a 5 μg/mL solution of purified recombinant human TLR-4/MD-2 with a 10× His-tag (extracellular N-terminal fragment, R&D Systems, USA, carrier-free) for 15 minutes, followed by another rinse with water. After each new layer of the mSAM (FIG. 1a) was deposited, the electrode was tested electrochemically using the same setup as used in the electrochemical experiments after SAM deposition, described above.

Sensor Testing & Circuit Fitting: TLR-4 modified working electrodes were exposed to concentrations of lipopolysaccharide (LPS, Sigma-Aldrich, serotype 0127:B8, phenol extracted) from 1 ng/mL to 10 μg/mL using the electrochemical methodology described above. The LPS was added to the cell via micropipette from concentrated stocks and mixed vigorously before being allowed to equilibrate for 20 minutes prior to electrochemical testing. The EIS response at the E^{0′} of the Fe-EDTA redox couple was recorded (10 mV rms amplitude and between 1 MHz and 10 mHz, sweeping in both directions) and the data were fitted to an equivalent circuit in order to obtain the best fit for the resistance across the interface. The EC-Lab software was used to approximate a fit and the resistance at each concentration of LPS was compared to the baseline levels for that electrode.

For the determination of the sensor response to gram-positive or gram-negative organisms, the electrodes were fabricated as described above and exposed to either heat inactivated Salmonella typhimurium (Invivogen) or heat killed Staphylococcus aureus (Invivogen) at concentrations ranging from 10⁰ cells/mL to 10⁵ cells/mL. All stock solutions of bacteria were thoroughly mixed prior to dilution to ensure homogeneity. Viral tests were conducted with UV-inactivated Rhabdovirus from an in-house preparation at 10⁴ and 10⁵ viral particles/mL (Cumming School of Medicine, University of Calgary).

Construction of modified SAMs: It has been shown that alkanethiols of shorter chain length (e.g., propanethiol) exhibit a lower impedance when deposited as a SAM on a Au electrode than longer alkanethiols, such as 11-mercaptoundecanoic acid (Campuzano et al., 2006). Previous studies have shown that a SAM composed of a mixture of two different thiols is capable of binding proteins with a polyhistidine tag. See, for example, Bonroy et al., 2006; Rickert et al., 1996. The present inventors have discovered that using shorter thiols as low-impedance “spacers” between longer chain and more resistive thiols, it was possible to provide a significantly more selective and sensitive sensors with a lower overall impedance. As an initial test, the present inventors fabricated a Au substrate with a mixture of 11-mercaptoundecanoic acid and two shorter thiols, a three-carbon 1-propanethiol (C3) and a five-carbon 1-pentanethiol (C5), and measured impedance.

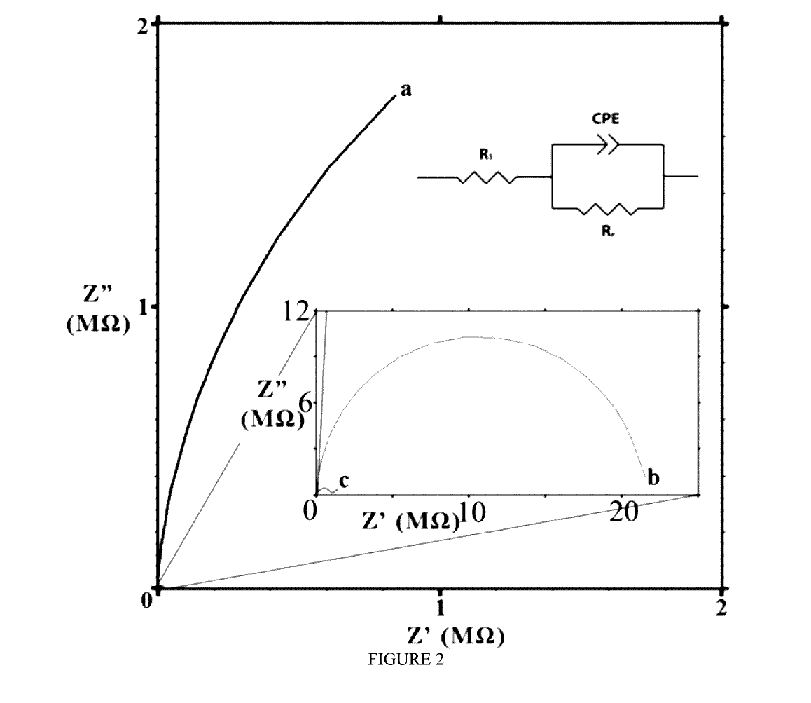

The observed impedance displayed a single time constant that represents the interface between the modified Au electrode and the solution. The data were approximated with a parallel interfacial resistor and constant phase element (CPE), with the solution resistance in series (FIG. 2). The n value associated with the CPE indicates its nature, with a value of 1 representing a pure, capacitor, 0 representing a resistor, and 0.5 typical of diffusion control (Warburg element). The n values in this work were typically in the range of 0.8 to 0.9, with no Warburg elements observed. Rp reflects the rate of the electrochemical reaction (Fe^{2+/3+} oxidation/reduction) and can be determined from the diameter of the impedance arc.

Initial experiments confirmed that the shorter chain alkanethiols generate smaller Rp values than SAMs of MUA alone, with a 24 hour propanethiol SAM providing only about 1Ω of resistance (FIG. 2). As shown in Table 1, a densely packed MUA SAM generates a much larger impedance, due to the nearly complete blockage of the Fe^{2+/3+} reaction occurring between or through the pentanethiol and propanethiol units in the SAM. By mixing the shorter thiols with the carboxylic acid containing MUA, it was anticipated that the overall impedance of the sensor would be lowered, thus amplifying the current response when analyte is present.

TABLE 1
Rₚ values of single and mixed thiol
SAMs after 24 hour deposition on Au

| SAM | Rₚ (Ω) |
|---|---|
| 11-mercaptoundecanoic acid (MUA) | 1.2 × 10⁶ |
| 1-Propanethiol (C3) | 9.6 × 10² |
| 1-Pentanethiol (C5) | 2.1 × 10⁴ |
| 9:1 C3:MUA | 6.7 × 10⁵ |
| 9:1 C5:MUA | 7.2 × 10⁵ |

* All Au substrates were cleaned, as outlined above, and then exposed to 10 mM total thiol for 24 hours in an ethanolic solution. Impedance data were collected at the E^{0′} of the Fe^{2+/3+} probe in solution and fitted to the circuit diagrammed in FIG. 2.

When these shorter chain thiols were mixed with MUA in a 9:1 molar ratio of spacer (C3 or C5) to MUA, followed by the overnight deposition process from this solution, Rp was observed to have become lower, as anticipated. As shown in Table 1, Rp after a 24 hour exposure of a clean Au substrate to 10 mM MUA is larger than that for an electrode coated with either the 9:1 molar ratio of C5:MUA or 9:1 C3:MUA SAMs. As expected, the C3/MUA monolayer resulted in a smaller Rp than that of the C5/MUA mixed SAM. Without being bound by any theory, it is believed that this is due to the ease of electron tunneling through the thinner (shorter thiol chains) parts of the film. See, for example, Kaur et al., 2013. It is also believed that the addition of the two extra carbons in the C5/MUA layer decreases the rate of electron tunneling when compared to the C3/MUA mix, hence the larger Rp. It is expected that neither spacer should provide steric hindrance to the reactions needed to immobilize the NTA functional group on the COOH group attached to MUA due to their small size and ease of stacking within the ordered matrix of the MUA alkane chains.

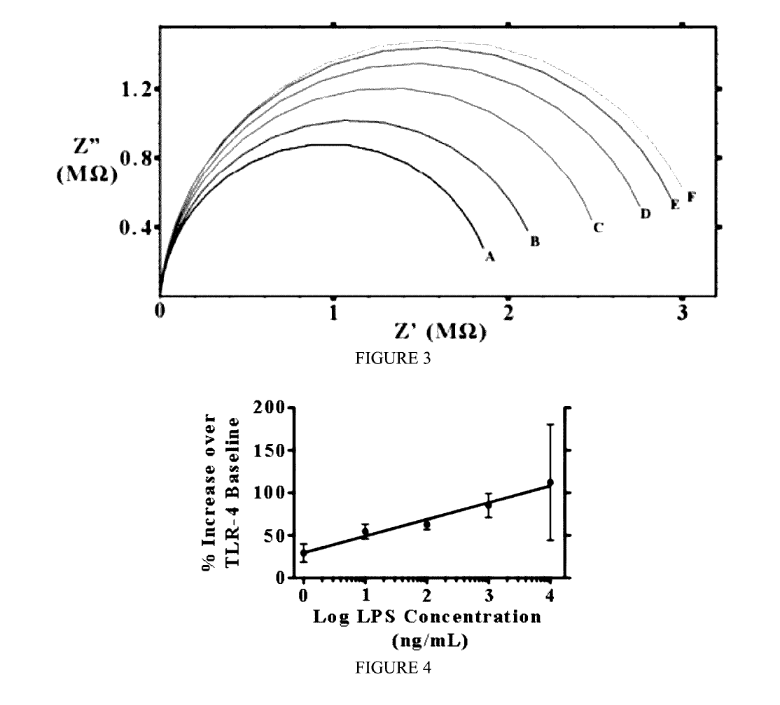

While the attachment of TLR-4 onto the outer surface of the mSAM via the histidine/Ni-NTA interaction brings the impedance of the mSAM to levels higher than that of MUA alone, the mixed mSAMs lower the overall impedance of the film to reasonable values. This arises from the shorter, and therefore lower resistance, pathways between the MUA thiols due to the incorporation of the C3 thiol, forming channels or ‘pores’ for the Fe^{2+/3+}-EDTA species to diffuse from the bulk solution to the underlying Au surface (FIG. 1). As can be seen in FIG. 3, the addition of LPS to the solution results in an increase in the electrode resistance proportionally to the amount of LPS added to the solution. This is believed to be due to the dimerization of TLR-4, bridging the MUA-based mSAMs above the C3 spacers (FIG. 1A) and effectively decreasing the size of these channels, as proposed in FIG. 1C. This lowers the overall rate of electron tunneling through the C3 thiols, thus increasing the measured resistance.

FIG. 4 shows the steady-state Rp of multiple sensors, constructed using the 9:1 molar ratio of the C3 thiol:MUA, obtained after the addition of LPS, with each bar representing the mean of the response of five different electrodes and the error bars showing the standard error of the mean. While some fluctuation in the resistance of the TLR-4 modified mSAMs is observed between samples (i.e., pre-LPS additions), likely partly related to variations in the sputtered Au electrode surface crystallinity, resulting in imperfections in the SAM structure/composition (Love et al., 2005), the percent change in the resistance due to the interactions with LPS remains comparable at most concentrations. At 10 μg/mL of LPS, however, increased variation is observed in the impedance response, suggesting an upper concentration limit for this sensor, coinciding with the maximum recommended concentration of LPS for TLR-4 activation in vivo, according to a major immunology retailer (InvivoGen).

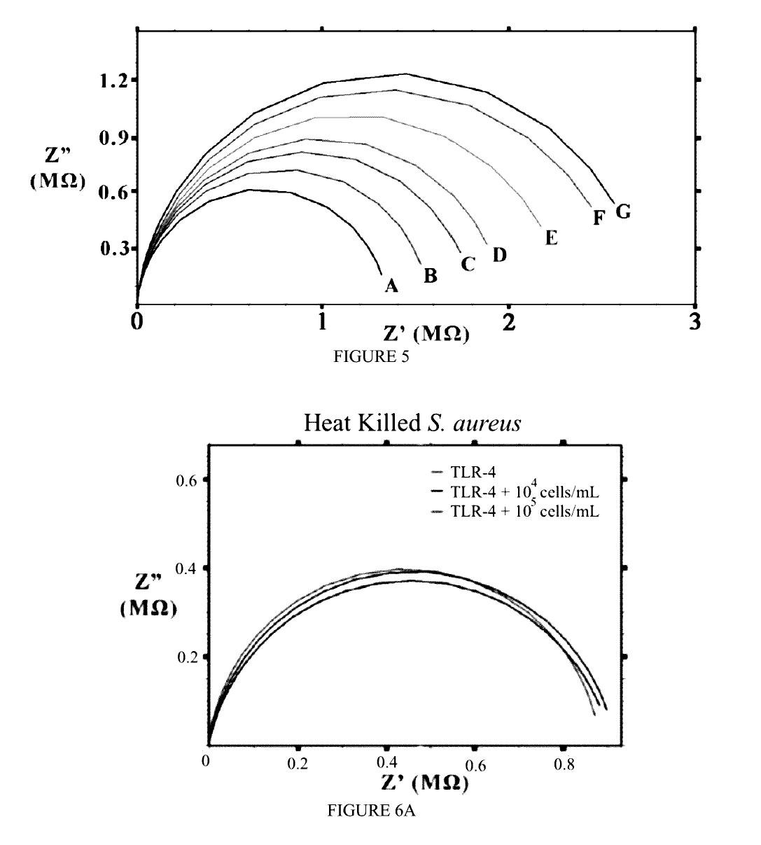

Inactive Organism Testing for Sensitivity and Specificity: The sensor was exposed to varying concentrations of the gram-negative organism Salmonella typhimurium. As shown in FIGS. 5 and 7, additions of Salmonella result in increases in the impedance of the Au electrode interface in a similar manner to that of an increasing solution concentration of purified LPS. However, with minimal detection occurring at 10⁰ cells/mL of Salmonella, the sensor exhibits greater sensitivity than to that of purified LPS. Assuming a molar mass of 100 kDa for LPS and based upon the levels of LPS expressed on the surface of Salmonella (Smit et al., 1975), 10° cells/mL corresponds to ca. 0.2 pg/mL of LPS in solution.

In particular as shown in FIG. 7, for a mSAM-based TLR-4 sensing layer deposited on a Au microelectrode (geometric area of 0.002 cm²), the addition of gram-negative bacteria caused a large resistance increase from the baseline (line b in FIG. 7), followed by a logarithmic increase in resistance with the further addition of Gram-negative bacteria (lines c and d in FIG. 7). This response is consistent with what was observed with the larger area electrodes treated with LPS or Gram-negative bacteria (0.5 cm²), and demonstrates that the TLR-4 modified mSAM can be used in sensing devices.

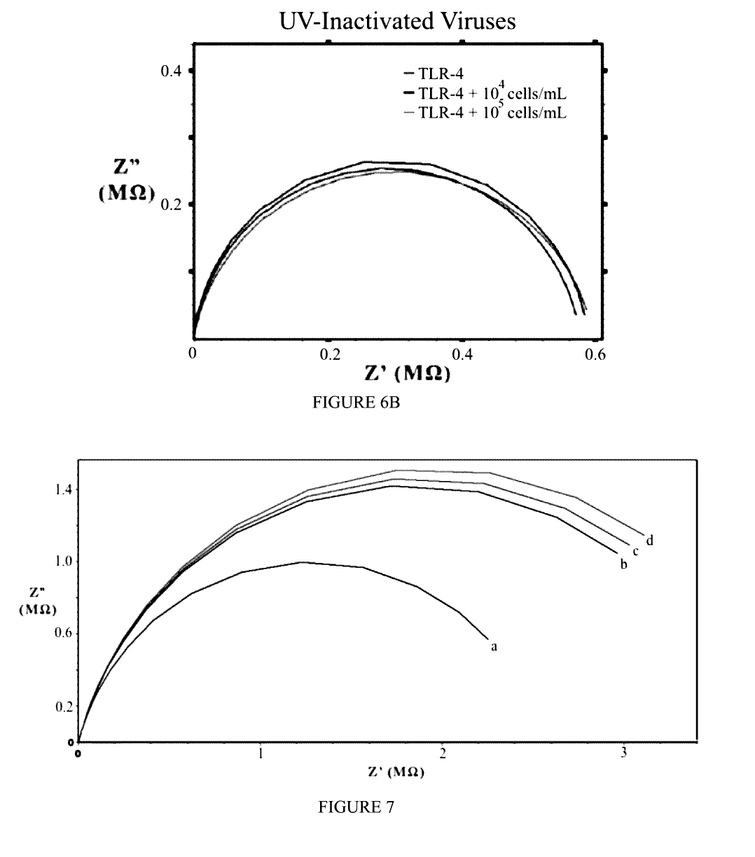

When exposed to the gram-positive bacterium Staphylococcus aureus at the high concentrations of 10⁴ and 10⁵ cells/mL, the sensor showed no significant sensitivity (FIG. 6A), as these bacteria do not produce surface LPS and therefore should not elicit any significant response from TLR-4. The same trend was also observed when the TLR-4 modified mSAMs were exposed to similar concentrations of viral particles, as shown in FIG. 6B. Coupled with the strong responses to ultra-low concentrations of the gram-negative Salmonella in solution, this clearly indicates that this sensor design is suitable for sensing gram-negative bacteria.

Insights into mechanism of LPS sensing by TLR-4 coated electrode: To further probe the mechanism by which the present sensor responds to LPS, the interaction of LPS with the electrode was examined at various stages of construction of the mSAM. Table 2 show the EIS circuit parameters obtained for clean Au, a 9:1 molar ratio of propanethiol:MUA, and a Ni-NTA functionalized mSAM to 10 μg/mL of LPS, all in the Fe³⁺-containing PBS. While the impedance of bare Au did increase slightly with the addition of LPS to the solution, which may be attributable to nonspecific physical adsorption processes, the impedance does not increase when the thiols or Ni-NTA layers are exposed to LPS. This indicates that the LPS sensing functionality is primarily due to the presence of TLR-4 on top of the mSAM, as when TLR-4 is not present, there is essentially no increase in the resistance when LPS is added to the solution.

TABLE 2
Effect of LPS on polarization resistance
of various mSAM-modified Au Electrodes*

| Substrate | Rₚ ( ) | % Change in Rₚ |
|---|---|---|
| Bare Au | 3.8 × 10² | — |
| Au-LPS | 4.1 × 10² | +6.0% |
| 9:1 C3:MUA | 6.7 × 10⁵ | — |
| 9:1 C3:MUA - LPS | 6.7 × 10⁵ | −0.2% |
| Ni-NTA | 3.0 × 10⁶ | — |
| Ni-NTA - LPS | 1.9 × 10⁶ | −35.5% |

*All samples were cleaned as discussed herein and then exposed to 10 mM total thiol for 24 hours in an ethanolic solution. C3 represents 1-protanethiol, which was mixed with 11-mercaptoundecanoic acid (MUA) in the construction of all SAMs. After formation of the SAM, the electrodes were then submerged in a mixture of 1-ethyl-3-(3-dimethylaminopropyl)carbodiimide and N-hydroxysuccinimide, followed by a subsequent incubation in carboxymethyl-L-lysine and NiSO₄ to form a Ni²⁺-nitrilotriacetic acid (Ni-NTA) functional group, as outlined herein. The response to LPS was conducted at 10 μg/mL to compare with the TLR-4 modified electrode. Impedance data were collected at the E^{0′} of the Fe^{2+/3+} probe in solution and fitted to the circuit shown in FIG. 2.

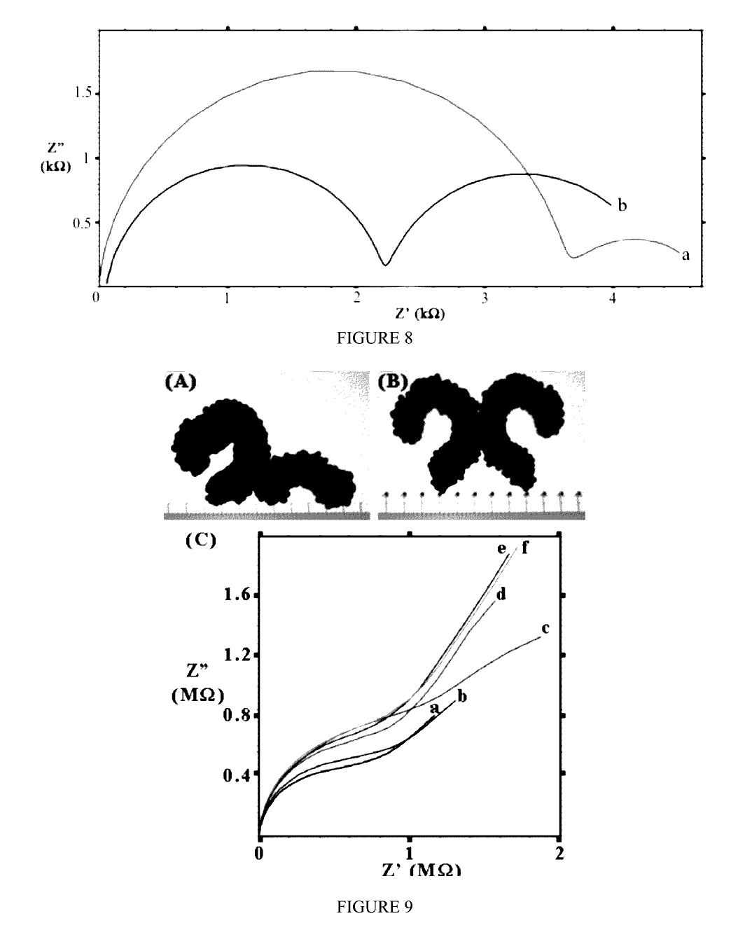

To determine if TLR-4 alone is sufficient to generate a response to LPS, TLR-4 was adsorbed onto a clean Au electrode by pipetting about 100 μL of 5 μg/mL TLR-4 onto the electrode surface and allowing the aliquot to dry in air. This was expected to deposit TLR-4 in a random manner on the surface, with no bonds holding the protein to the electrode surface. This should result in a minimal LPS response, as there would be no organized channels that could be closed due to a dimerization event. As predicted, the EIS response (FIG. 8) was significantly different from the results shown in FIG. 3 for the sensor in which TLR-4 was bound in a controlled orientation on the modified SAM substrate. The physisorbed TLR-4 gives two time constants (two arcs in the Nyquist plot in FIG. 8) versus the previous single time constant (FIGS. 3, 5, and 6).

When exposed to 1 μg/mL LPS, the relative resistance of the two time constants is seen to change. However, the total resistance (full diameter of both arcs in FIG. 8) did not increase significantly.

While physisorbed TLR-4 is not sufficient to generate a response to LPS, the interaction of TLR-4 immobilized to the mixed SAM was also probed in a random orientation (FIGS. 9A and 9B). By activating the terminal COOH group of MUA with the EDC/NHS method and then exposing the electrode to TLR-4 for 24 hours at 4° C., TLR-4 was immobilized to a SAM composed of a 9:1 molar ratio of 1-propanethiol:MUA in a disordered manner (i.e., any primary amine on the protein could interact with the activated COOH to immobilize the protein). As expected, the response to LPS was weak, with only a slight increase in the resistance seen as the LPS concentration was increased, without a linear response observed to variable LPS concentrations.

FIG. 9C shows the response of the direct coupling to TLR-4 to a 9:1 molar ratio of C3:MUA SAM on Au. As can be seen, the response was not proportional to the amount of LPS added. Similar results, where an orientated protein provides a larger response than a randomly oriented control, have been reported in the literature previously (Bonroy et al., 2006). This negative outcome makes randomly orientated TLR-4 deposited onto a SAM a poor choice as a LPS sensor.

One interesting trend is that the mSAM/TLR-4 modified electrodes respond to LPS or bacteria in a logarithmic fashion. This is consistent with a LPS-TLR-4 interaction that obeys a surface adsorption isotherm, such as is reported for the Temkin isotherm (Johnson and Arnold, 1995). If this isotherm is being followed here, this would indicate that, as more LPS is bound onto the surface, the equilibrium constant defining the binding of LPS to TLR-4 is lowered, reflecting repulsive lateral interactions between surface LPS groups. The belief that the binding constant should change as more TLR-4 is bound is supported by attempting to visualize a field of TLR-4 moieties on a surface, as if one pair of TLR-4 molecules dimerizes, their neighbors lose a potential partner for their own dimerization with LPS.

An alternative, biological, rationale for the logarithmic dependence of the measured resistance on the LPS concentration is related to the role of TLR-4 in the human body. As this protein is responsible for triggering a signaling cascade that could cause systemic inflammation (i.e., sepsis), TLR-4 must be carefully regulated. By exhibiting a response to only large shifts in the concentration of LPS, TLR-4 would ration the potentially lethal response to sepsis that could otherwise be triggered by the innate immune system.

One particular sensor discussed herein was constructed by the initial deposition of a self-assembled monolayer (SAM) and then attaching a nickel nitrilotriacetic acid (Ni-NTA) moiety, followed by a poly-histidine tagged Toll-Like Receptor-4 (TLR-4) to form a modified SAM (mSAM) layer. Without being bound by any theory, it is believed that the sensitivity and selectivity of the sensor of the invention is due at least in part to the binding of LPS or gram-negative bacteria to TLR-4, thereby causing TLR-4 dimerization on the surface, thus partially closing off channels within the mSAM. The sensor was examined in a pH 7 solution typically containing 5 mM of Fe³⁺-EDTA, and the rate of the Fe^{2+/3+} redox reaction was then tracked as a function of LPS concentration. A 9:1 molar ratio of short thiols (1-propanethiol) to long, derivatizable thiols (11-mercaptoundecanoic acid) was shown to lower the overall impedance of the modified electrode to that of levels tolerable by portable, low-cost electrochemical instruments. This sensor showed a reproducible, logarithmic, dose-dependent increase in the impedance when aliquots of LPS were added to the supporting electrolyte. This fits with the predictions of the Temkin isotherm, which has previously been shown to be relevant to biological systems. The logarithmic response was also seen when the sensor was challenged with gram-negative bacteria, while no response was observed when it was exposed to gram-positive bacteria or to viral particles.

The sensor was also shown to be highly dependent on the incorporation of a flexible alkanethiol linker between the protein and the Au surface, as when there was no SAM present or the protein was immobilized on the surface in a random manner, the sensor response was greatly diminished. These results are consistent with the prediction of model, in which Fe^{3+/2+} ions can react at the short spacer thiols between the larger thiols, essentially creating channels between the Au electrode and the bulk solution. As this model depends on the inducible dimerization of a protein, this sensing strategy can be applied to any protein-ligand interaction with this characteristic, including other immune system receptors.

Detection of gram-negative bacteria: When bacteria, including gram-negative bacteria, are airborne they are encapsulated within a drop of water or aerosol of moisture. Aerosols in the air sample can be collected in to the buffer solution described herein such that these bacteria are collected in the fluid sample and could then be detected using the apparatus of the invention. Any of the conventional methods for collecting air or aerosol sample for analysis can be used.

While there is typically always some background level of bacteria, if a large amount of gram-negative bacteria is released intentionally or unintentionally as an airborne pathogenic agent (e.g., as a biological warfare agent), the amount of gram-negative bacteriain the air sample will be increased substantially compared to baseline amounts, i.e., “normal” conditions where no gram-negative bacteria was intentionally or unintentionally released by an outside agent, such as human.

The apparatus of the invention can be used in indoor settings, such as in hospitals and other areas public areas, where the “baseline” levels are expected to be somewhat more consistent, i.e., no “background” measurement would be needed. In some methods, a multiplexed array of the apparatuses of the invention can be used to perform a measurement every few hours to collect background “noise level”, i.e., ambient amount of gram-negative bacteria present in the area.

Such information is useful if there was a bioterrorism event, as it would allow for quick quarantine of affected areas by identifying them sooner than current methods. Methods and apparatuses of the invention can also be usee for detecting a large amount of gram-negative bacteria in open environment.

Bacterial capture and concentration from aerosol into a liquid sampling chamber can be achieved through several strategies. The simplest strategy is to us a vacuum to pass air samples into a solution. While this strategy can be used, it is believed that this sampling process does not efficiently direct or concentrate bacteria onto a surface. It should be appreciated that any method of collecting a sample for bacteria detection known to one skilled in the art can be used, including swabbing a sample to be tested, obtaining a fluid sample (e.g., blood, saliva, mucous of a subject), etc. can be used for collecting a sample for testing. With regards to air sampling, the following is two exemplary strategies to selectively concentrate bacteria obtained from air particles.

Bacterial aerosols are often charged. Electrostatic detection (Wei et al. 2014) involves collection of bacterial aerosols by passage of particles through a chamber where an electric charge is applied (chamber is lined with oppositely charged copper plates). Aerosols are collected against a filter according to preferential charge interaction. See FIG. 10a. This method is selective for bacterial aerosols that are charged, being independent of particle size. The filters can then be rinsed through a flow system into the sampling chamber with TLR-4 (FIG. 10c).

Another strategy for aerosol collection is through acoustic channeling (Yuen et al. 2014). The principle of this concentration strategy is to bombard air particles with acoustic waves from the walls of a collection chamber to directionally channel particles to a membrane or liquid sampling chamber (FIG. 10b). This strategy works well with particles between 0.3 to 6 micron sizes and it does not have a bias for charged particles. The particles are then eluted and detected in the same manner as in the electrostatic detection.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

### CITED REFERENCES

Abdul Rahman, M. S., Mukhopadhyay, S. C., Yu, P. L., Goicoechea, J., Matias, I. R., Gooneratne, C. P., Kosel, J., 2013. Detection of bacterial endotoxin in food: New planar interdigital sensors based approach. J. Food Eng. 114, 346-360. doi:10.1016/j.jfoodeng.2012.08.026
Amini, K., Chan, N. W. C., Kraatz, H.-B., 2014. Toll-like receptor 3 modified Au electrodes: an investigation into the interaction of TLR3 immobilized on Au surfaces with poly(I:C). Anal. Methods 6, 3322. doi:10.1039/c4ay00369a
Besant, J. D., Das, J., Sargent, E. H., Kelley, S. O., 2013. Proximal bacterial lysis and detection in nanoliter wells using electrochemistry. ACS Nano 7, 8183-8189. doi:10.1021/nn4035298
Beutler, B., 2000. Tlr4: Central component of the sole mammalian LPS sensor. Curr. Opin. Immunol. 12, 20-26. doi:10.1016/S0952-7915(99)00046-1
Bloos, F., 2015. Clinical Diagnosis of Sepsis and the Combined Use of Biomarkers and Culture- and Non-Culture-Based Assays, in: Mancini, N. (Ed.), Sepsis: Diagnostic Methods and Protocols. pp. 247-260. doi:10.1007/978-1-4939-1776-1.
Bonroy, K., Frederix, F., Reekmans, G., Dewolf, E., De Palma, R., Borghs, G., Declerck, P., Goddeeris, B., 2006. Comparison of random and oriented immobilisation of antibody fragments on mixed self-assembled monolayers. J. Immunol. Methods 312, 167-181. doi:10.1016/j.jim.2006.03.007
Campuzano, S., Pedrero, M., Montemayor, C., Fatás, E., Pingarrón, J. M., 2006. Characterization of alkanethiol-self-assembled monolayers-modified gold electrodes by electrochemical impedance spectroscopy. J. Electroanal. Chem. 586, 112-121. doi:10.1016/j.jelechem.2005.09.007
Canaria, C. a, So, J., Maloney, J. R., Yu, C. J., Smith, J. O., Roukes, M. L., Fraser, S. E., Lansford, R., 2006. Formation and removal of alkylthiolate self-assembled monolayers on gold in aqueous solutions. Lab Chip 6, 289-295. doi:10.1039/b510661c
Das, J., Kelley, S O., 2013. Tuning the bacterial detection sensitivity of nanostructured microelectrodes. Anal. Chem. 85, 7333-7338. doi:10.1021/ac401221f
Diao, P., Guo, M., Tong, R., 2001. Characterization of defects in the formation process of self-assembled thiol monolayers by electrochemical impedance spectroscopy. J. Electroanal. Chem. doi:10.1016/S0022-0728(00)00424-1
Ding, S. J., Chang, B. W., Wu, C. C., Chen, C. J., Chang, H. C., 2007. A new method for detection of endotoxin on polymyxin B-immobilized gold electrodes. Electrochem. commun. 9, 1206-1211. doi:10.1016/j.elecom.2006.12.029
Gronbeck, H., Curioni, A., Andreoni, W., 2000. Thiols and Disulfides on the Au (111) Surface: The Headgroup—Gold Interaction. J. Am. Chem. Soc. 122, 3839-3842. doi:10.1021/ja993622x
Han, A., Takarada, T., Shibata, T., Nakayama, M., Maeda, M., 2006. A MutS protein-immobilized au electrode for detecting single-base mismatch of DNA. Anal. Sci. 22, 663-666. doi:10.2116/analsci.22.663
Hochuli, E., Döbeli, H., Schacher, a, 1987. New metal chelate adsorbent selective for proteins and peptides containing neighbouring histidine residues. J. Chromatogr. 411, 177-184. doi:10.1016/S0021-9673(00)93969-4
Iijima, S., Kato, D., Yabuki, S., Niwa, O., Mizutani, F., 2011. Enzymatically amplified electrochemical detection for lipopolysaccharide using ferrocene-attached polymyxin B and its analogue. Biosens. Bioelectron. 26, 2080-2084. doi:10.1016/j.bios.2010.09.009
Ivanov, I., Stojcic, J., Stanimirovic, A., Sargent, E., Nam, R. K., Kelley, S. O., 2013. Chip-based nanostructured sensors enable accurate identification and classification of circulating tumor cells in prostate cancer patient blood samples. Anal. Chem. 85, 398-403. doi:10.1021/ac3029739.
Johnson, R. D., Arnold, F. H., 1995. The Temkin isotherm describes heterogeneous protein adsorption. Biochim. Biophys. Acta—Protein Struct. Mol. Enzymol. 1247, 293-297. doi:10.1016/0167-4838(95)00006-G
Kato, D., Iijima, S., Kurita, R., Sato, Y., Jia, J., Yabuki, S., Mizutani, F., Niwa, O., 2007. Electrochemically amplified detection for lipopolysaccharide using ferrocenylboronic acid. Biosens. Bioelectron. 22, 1527-1531. doi:10.1016/j.bios.2006.05.020
Kaur, I., Zhao, X., Bryce, M. R., Schauer, P. a., Low, P. J., Kataky, R., 2013. Modification of electrode surfaces by self-assembled monolayers of thiol-terminated oligo(Phenyleneethynylene)s. ChemPhysChem 14, 431-440. doi:10.1002/cphc.201200744
Kim, S. E., Su, W., Cho, M., Lee, Y., Choe, W. S., 2012. Harnessing aptamers for electrochemical detection of endotoxin. Anal. Biochem. 424, 12-20. doi:10.1016/j.ab.2012.02.016
Kröger, D., Liley, M., Schiweck, W., Skerra, A., Vogel, H., 1999. Immobilization of histidine-tagged proteins on gold surfaces using chelator thioalkanes. Biosens. Bioelectron. 14, 155-161. doi:10.1016/S0956-5663(98)00116-X
Love, J. C., Estroff, L. A., Kriebel, J. K., Nuzzo, R. G., Whitesides, G. M., 2005. Self-Assembled Monolayers of Thiolates on Methals as a Form of Nanotechnology.
Neu, H. C., 1992. The crisis in antibiotic resistance. Science 257, 1064-1073. doi:10.1126/science.257.5073.1064
Nicosia, C., Huskens, J., 2014. Reactive self-assembled monolayers: from surface functionalization to gradient formation. Mater. Horizons 1, 32. doi:10.1039/c3mh00046j
Park, B. S., Song, D. H., Kim, H. M., Choi, B.-S., Lee, H., Lee, J.-O., 2009. The structural basis of lipopolysaccharide recognition by the TLR4-MD-2 complex. Nature 458, 1191-1195. doi:10.1038/nature07830
Priano, G., Pallarola, D., Battaglini, F., 2007. Endotoxin detection in a competitive electrochemical assay: Synthesis of a suitable endotoxin conjugate. Anal. Biochem. 362, 108-116. doi:10.1016/j.ab.2006.12.034
Rickert, J., Göpel, W., Beck, W., Jung, G., Heiduschka, P., 1996. A “mixed” self-assembled monolayer for an impedimetric immunosensor. Biosens. Bioelectron. 11, 757-768. doi:10.1016/0956-5663(96)85927-6.
Smit, J., Kamio, Y., Nikaido, H., 1975. Outer membrane of Salmonella typhimurium: chemical analysis and freeze-fracture studies with lipopolysaccharide mutants. J. Bacteriol. 124, 942-958.
Su, W., Kim, S.-E., Cho, M., Nam, J.-D., Choe, W.-S., Lee, Y., 2013. Selective detection of endotoxin using an impedance aptasensor with electrochemically deposited gold nanoparticles. Innate Immun. 19, 388-97. doi:10.1177/1753425912465099
Su, W., Lin, M., Lee, H., Cho, M., Choe, W. S., Lee, Y., 2012. Determination of endotoxin through an aptamer-based impedance biosensor. Biosens. Bioelectron. 32, 32-36. doi:10.1016/j.bios.2011.11.009
Witt, D., Klajn, R., 2004. Applications, properties and synthesis of omega-functionalized n-alkanethiols and disulfides—the building blocks of self-assembled monolayers. Curr. Org. Chem. 8, 1-34. doi:10.2174/1385272043369421
Yeo, T. Y., Choi, J. S., Lee, B. K., Kim, B. S., Yoon, H I., Lee, H. Y., Cho, Y. W., 2011. Electrochemical endotoxin sensors based on TLR4/MD-2 complexes immobilized on gold electrodes. Biosens. Bioelectron. 28, 139-145. doi:10.1016/j.bios.2011.07.010.
Wei, K., Zou, Z. & Yao, M. Charge levels and Gram (7) fractions of environmental bacterial aerosols. 74, 52-62 (2014).
Yuen, W. T. et al. The Use of Nonlinear Acoustics as an Energy-Efficient Technique for Aerosol Removal The Use of Nonlinear Acoustics as an Energy-Efficient Technique for Aerosol Removal. 6826, (2014)

What is claimed is:

1. A solid substrate comprising:
an electric conducting solid substrate surface having a monolayer of a mixture of linkers each of which has a first functional group that is attached to the surface of said electric conducting solid substrate, wherein said mixture of linkers comprises a tethering-linker and a spacer-linker in a ratio of at least 1:1, and wherein said tethering linker comprises a second functional group; and
a toll-like receptor 4 (TLR-4) that is attached to said tethering linker, wherein the chain length of said spacing-linker is smaller than the chain length of said tethering-linker.

2. The solid substrate of claim 1, wherein the ratio of said spacer-linker to said tethering-linker is at least 5:1.

3. The solid substrate of claim 1, wherein the chain length of said tethering-linker is at least 3 atoms longer than the chain length of said spacer-linker.

4. The solid substrate of claim 1, wherein said TLR-4 is attached to said tethering-linker through a metal cation that is coordinated to said second functional group.

5. The solid substrate of claim 4, wherein said second function group of said tethering-linker comprises nitrilotriacetic acid (NTA), and wherein said metal cation forms a metal-NTA coordinated complex.

6. The solid substrate of claim 5, wherein said TLR-4 is attached to said tethering-linker via a coordination of a polyhistidine group of said TLR-4 to said metal-NTA coordinated complex.

7. The solid substrate of claim 6, wherein said metal comprises Ni⁺².

8. The solid substrate of claim 1, wherein said TLR-4 further comprises lymphocyte antigen 96 (MD-2).

9. The solid substrate of claim 8, wherein said TLR-4 is a recombinant human TLR-4/MD-2.

10. The solid substrate of claim 9, wherein said TLR-4 further comprises a polyhistidine tag.

11. A method for detecting the presence of a gram-negative bacteria in a sample, said method comprising:
(i) a sample in an apparatus comprising a solid substrate of claim 1 under conditions sufficient to allow a gram-negative bacteria, if present in said sample, to attach to said solid substrate;
(ii) placing the resulting solid substrate of said step (i) in a redox solution; and
(iii) measuring the impedance within said solid substrate to determine the presence of a gram-negative bacteria,
wherein change in impedance compared to a baseline impedance of said solid substrate is an indication that a gram-negative bacteria is present in said sample.

12. The method of claim 11, wherein said TLR-4 is attached to said tethering-linker through a metal cation that selectively binds to a poly-histidine portion of said TLR-4, and wherein said metal cation forms a coordinating complex with said second functional group of said tethering-linker.

13. The method of claim 12, wherein said second function group of said tethering-linker comprises nitrilotriacetic acid (NTA), and wherein said metal cation forms a metal-NTA coordinated complex.

14. The method of claim 13, wherein said TLR-4 is attached to said tethering-linker via a coordination of a polyhistidine group of said TLR-4 to said metal-NTA coordinated complex.

15. The method of claim 14, wherein said metal comprises Ni⁺².

16. The method of claim 11, wherein said TLR-4 further comprises lymphocyte antigen 96 (MD-2).

17. The method of claim 16, wherein said TLR-4 is a recombinant human TLR-4/MD-2.

18. The method of claim 17, wherein said TLR-4 further comprises a polyhistidine tag.

19. A method for producing a solid substrate comprising:
an electric conducting solid substrate surface having a monolayer of a mixture of linkers each of which has a first functional group that is attached to the surface of said electric conducting solid substrate, wherein said mixture of linkers comprises a tethering-linker and a spacer-linker in a ratio of x:1, wherein x is a number from 1 to 10, and wherein said tethering linker further comprises a second functional group; and
a toll-like receptor 4 (TLR-4) that is attached to said tethering linker at said second functional group;
said method comprising:
(i) contacting an electric conducting solid substrate with a solution comprising a mixture of linkers each of which has a first functional group, said mixture of linkers comprising a tethering-linker and a spacer-linker in a ratio of x:1 under conditions to form a monolayer of mixture of linkers wherein said first functional group is attached to the surface of said electric conducting solid substrate at a ratio of x:1 between said tethering-linker and said spacer-linker; and
(ii) attaching a toll-like receptor 4 (TLR-4) in an orderly manner onto said second functional group of said tethering-linker.

20. The method of claim 19 further comprising the steps of:
converting said second functional group of said tethering-linker to a polyhistidine coordinating complex; and
attaching a polyhistidine portion of said TLR-4 to said polyhistidine coordinating complex.
